(19) United States
(12) Patent Application Publication
(10) Pub. No.: US 2005/0049255 A1
(43) Pub. Date: Mar. 3, 2005

(54) Therapeutic combinations

(75) Inventors: Magda Nabil Bictash, Sandwich (GB); Rachel Jane Russell, Sandwich (GB); Piet Hadewijn VanDerGraaf, Sandwich (GB); Christopher Peter Wayman, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/865,194

(22) Filed: Jun. 10, 2004

Related U.S. Application Data
(60) Provisional application No. 60/484,266, filed on Jun. 30, 2003.

(30) Foreign Application Priority Data
Jun. 10, 2003 (GB) 0313363.4

(51) Int. Cl. A61K 31/519

(52) U.S. Cl. 514/252.16; 514/262.1

(57) ABSTRACT

Synergistic combinations of antagonists of the vasopressin receptor family with PDE inhibitors are described.

This application claims priority to U.S. Provisional Application Ser. No. 60/484,266, filed Jun. 30, 2003, which claims priority to British Application Serial No. GB 0313363.4, filed Jun. 10, 2003.

This invention relates to a synergistic combination of antagonists of the vasopressin receptor family with PDE inhibitors, the use of such combinations in the treatment of dysmenorrhoea, methods of treating dysmenorrhoea using such combinations, and medicaments containing such combinations.

There is a high unmet need in the area of menstrual disorders and it is estimated that up to 90% of all menstruating women are affected to some degree. Up to 42% of women miss work or other activities due to menstrual pain and it has been estimated that around 600 million work hours a year are lost in the US as a result {Coco, A. S. (1999). Primary dysmenorrhoea. [Review] [30 refs]. American Family Physician, 60, 489-96.}.

Dysmenorrhoea can be divided into two classes, primary and secondary. Primary dysmenorrhoea is generally defined as cramping pain in the lower abdomen occurring at the onset of menstruation, in the absence of any identifiable pelvic disease. This affects approximately 50% of the female population {Coco, A. S. (1999). Primary dysmenorrhoea. [Review] [30 refs]. American Family Physician, 60, 489-96.; Schroeder, B. & Sanfilippo, J. S. (1999). Dysmenorrhoea and pelvic pain in adolescents. [Review] [78 refs]. Pediatric Clinics of North America, 46, 555-71}.

Secondary dysmenorrhoea is described as painful menstruation associated with specific pathological conditions such as endometriosis, pelvic inflammatory disease, fibroids, intra-uterine contraceptive devices etc. The pathogenesis of dysmenorrhoea is unknown, although there appears to be a close association between myometrial hyperactivity and reduced uterine blood flow with the pain felt by these women. Secondary dysmenorrhoea is diagnosed in only approximately 25% of women suffering from dysmenorrhoea. Dysmenorrhoea can occur in conjunction with menorrhagia.

In healthy women uterine contractility varies during the menstrual cycle {Akerlund, M. (1997), Contractility in the non-pregnant uterus. [Review] Annals of the New York Academy of Sciences, 828, 213-22.). The changes do not follow the fluctuations in plasma concentrations of ovarian hormones, but may be related to tissue levels because there are time lags between the two. During the first few days of the menstrual cycle uterine contractility is coordinated throughout the whole uterus, with contractions that are regular and of comparatively high amplitude, with well-demarked relaxations between the contractions. During the follicular phase, in particular around the time of ovulation a more uncoordinated uterine contractility occurs, with contractions that are of relatively high frequency, low amplitude and high basal tone. This continues through the luteal phase until 2-3 days before the onset of menstruation when the uterine activity becomes more coordinated again. At this time the cyclic propagation of contractions occurs, both in the direction of the cervix and towards the fundus. The direction can change for a patient within a few minutes. Propagation towards the cervix may be important for the expulsion of endometrium and blood at menstruation.

In comparison, women with dysmenorrhoea have pronounced uterine hyperactivity. Their contractile patterns are irregular. Also uterine blood flow is reduced and is mainly ischaemic in nature {Akerlund, M. (1997), Contractility in the non-pregnant uterus. [Review] Annals of the New York Academy of Sciences, 828, 213-22.}. The reduction in blood flow is probably an effect of both:
Compression of vessels caused by increased uterine pressure—it is believed this may be associated with the colicky pain experienced by these women
An influence of vasoactive agents on the smooth muscle of arterial walls causing longer lasting reduction in blood flow—this may be cause of the continuous aching pain experienced by these women.

Of the currently available treatments for dysmenorrhoea, non-steroidal anti-inflammatory drugs (NSAID's) tend to be the first line choice unless birth control is also desired, in which case oral contraceptives are used.

Primary dysmenorrhoea has been associated with increased endometrial prostaglandin F_{2α} (PGF_{2α}) at the time of menstruation {Pickles, V., Hall, W. & Best, F. (1965). Prostaglandins in endometrium and menstrual fluid from normal and dysmenorrheic subjects. BJOG: an International Journal of Obstetrics & Gynecology, 72, 185-.}, but not perimenstruation {Lundstrom, V. & Green, K. (1978). Endogenous levels of prostaglandin F2alpha and its main metabolites in plasma and endometrium of normal and dysmenorrheic women. American Journal of Obstetrics & Gynecology, 130, 640-6.). PGF_{2α} is known to increase uterine contractility and cause dysmenorrhoeic like pain {Roth-Brandel, U., Bygdeman, M. & Wiqvist, N. (1970). Effect of intravenous administration of prostaglandin E1, and F2 on the contractility of the non-pregnant human uterus in vivo. Acta Obstetricia et Gynecologica Scandinavica—Supplement, 5, 19-25.}. Prostaglandins are also known to have direct pain-producing properties by sensitizing pain receptors, which may also be involved in the pain felt at the time of menstruation {Ferreira, S. (1976). Pain and Fever. In Prostaglandin and Thromboxanes: NATO advanced study institute on advances on prostaglandins. pp. 433-442. New York: Plenum Press.}. NSAID's have been shown in clinical trials to alleviate pain and restore uterine motility in some dysmenorrhoeic patients {Pulkkinen, M. O. & Csapo, A. I. (1978). The effect of ibuprofen on the intrauterine pressure and menstrual pain of dysmenorrheic patients. Prostaglandins, 15, 1055-62.}. However, they are not effective in all dysmenorrhoeic sufferers, in particular those with severe dysmenorrhoea. Furthermore, they are associated with side effects including upper gastrointestinal tract symptoms, drowsiness and tinnitus. These agents do have the advantage over oral contraceptives of only being administered for 2-3 days per month and they reduce some of the side effects associated with dysmenorrhoea (dizziness, nausea and vomiting).

Oral contraceptives are a second line therapy for most women unless birth control is also desired. They have to be taken continuously throughout the cycle and it may take up to 3 cycles for menstrual pain to noticeably diminish. In comparison to NSAID's, oral contraceptives prevent menstrual pain by reducing menstrual fluid volume {Nakano, R. & Takemura, H. (1971). Treatment of functional dysmenorrhoea; a double-blind study. Acta Obstetrica et Gynaecologica Japonica, 18, 41-4.}, suppressing ovulation and decreasing endometrial volume. Thus resulting in a decrease in prostaglandin production {Chan, W. Y. & Hill, J. C. (1978). Determination of menstrual prostaglandin levels in non-dysmenorrheic and dysmenorrheic subjects. Prostaglandins, 15, 365-75.}.

However, it is a recognized problem that there is a persistent failure rate with NSAID's and oral contraceptives (10-15%), particularly in patients with severe dysmenorrhoea {Coco, A. S. (1999). Primary dysmenorrhoea. [Review]. American Family Physician, 60, 489-96; Schroeder, B. & Sanfilippo, J. S. (1999). Dysmenorrhoea and pelvic pain in adolescents. [Review]. Pediatric Clinics of North America, 46, 555-71.}. Newer, less well-characterized treatments are now being investigated, but they are not yet available as a current therapy option. These include treatment with V_{1A} antagonists and NO donors.

The physiological response of the uterus to vasopressin changes throughout the menstrual cycle, with a maximal sensitivity observed pre-menstrually {Bossmar, T., Akerlund, M., Szamatowicz, J., Laudanski, T., Fantoni, G. & Maggi, M. (1995). Receptor-mediated uterine effects of vasopressin and oxytocin in non-pregnant women. British Journal of Obstetrics & Gynaecology, 102, 907-12.}. Plasma vasopressin levels do not alter significantly during the menstrual cycle of control patients {Forsling, M. L., Akerlund, M. & Stromberg, P. (1981). Variations in plasma concentrations of vasopressin during the menstrual cycle. Journal of Endocrinology, 89, 263-6.}. However, vasopressin levels are raised in dysmenorrhoeic women both pre- and during menstruation {Hauksson, A., Akerlund, M., Forsling, M. L. & Kindahl, H. (1987). Plasma concentrations of vasopressin and a prostaglandin F2 alpha metabolite in women with primary dysmenorrhoea before and during treatment with a combined oral contraceptive. Journal of Endocrinology, 115, 355-61.}. This occurs in association with increased pain, myometrial hyperactivity and reduced uterine blood flow {Ekstrom, P., Akerlund, M., Forsling, M., Kindahl, H., Laudanski, T. & Mrugacz, G. (1992). Stimulation of vasopressin release in women with primary dysmenorrhoea and after oral contraceptive treatment—effect on uterine contractility. British Journal of Obstetrics & Gynaecology, 99, 680-4.}. The exact details of the contribution of vasopressin in the mechanisms of menstruation and dysmenorrhoea are still unknown. However, the peptide has pronounced constrictor effects on smooth muscle activity of both myometrium {Bossmar, T., Brouard, R., Doberl, A. & Akerlund, M. (1997). Effects of SR 49059, an orally active V1a vasopressin receptor antagonist, on vasopressin-induced uterine contractions. British Journal of Obstetrics & Gynaecology, 104, 471-7.} and uterine arteries {Kostrzewska, A., Laudanski, T., Steinwall, M., Bossmar, T., Serradeil-Le Gal, C. & Akerlund, M. (1998). Effects of the vasopressin V1a receptor antagonist, SR 49059, on the response of human uterine arteries to vasopressin and other vasoactive substances. Acta Obstetricia et Gynecologica Scandinavica, 77, 3-7.} via the vasopressin V_{1A} receptor, which is different from the V₂ type regulating kidney function.

There is clinical evidence that an orally active V_{1A} antagonist has a significant therapeutic benefit in the prevention of dysmenorrhoea {Akerlund, M. (1987). Can primary dysmenorrhoea be alleviated by a vasopressin antagonist? Results of a pilot study. Acta Obstetricia et Gynecologica Scandinavica, 66, 459-61.}. In a double blind, randomised placebo controlled cross over trial, Relcovaptan (SR49059) dose dependently reduced the intensity of pain in women with dysmenorrhoea, without effecting the mechanisms regulating menstruation {Brouard, R., Bossmar, T., Fournie-Lloret, D., Chassard, D. & Akerlund, M. (2000). Effect of SR49059, an orally active V1a vasopressin receptor antagonist, in the prevention of dysmenorrhoea. BJOG: an International Journal of Obstetrics & Gynaecology, 107, 614-9.}. This compound also displays activity at the Oxytocin receptor. The peptide vasopressin V_{1A} antagonist/Oxytocin receptor antagonist 1-deamino-2-D-Tyr (Oet)-4-Thr-8-Orn-oxytocin, when given intravenously is also effective in the treatment of dysmenorrhoea {Akerlund, M. (1987). Can primary dysmenorrhoea be alleviated by a vasopressin antagonist? Results of a pilot study. Acta Obstetricia et Gynecologica Scandinavica, 66, 459-61.}.

Nitric oxide mediates smooth muscle relaxation and vasodilatation. Clinical studies have shown beneficial effects of NO donors on inhibiting myometrial contractility in both preterm labour {Lees, C., Campbell, S., Jauniaux, E., Brown, R., Ramsay, B., Gibb, D., Moncada, S. & Martin, J. F. (1994). Arrest of preterm labour and prolongation of gestation with glyceryl trinitrate, a nitric oxide donor.[comment]. Lancet, 343, 1325-6.} and dysmenorrhoea {Pittrof, R., Lees, C., Thompson, C., Pickles, A., Martin, J. F. & Campbell, S. (1996). Crossover study of glyceryl trinitrate patches for controlling pain in women with severe dysmenorrhoea. Bmj, 312, 884.}. However, side effects are reported with NO donors, the most common being headache, dizziness and flushing {Ghazizadeh, S., Dadkhah, T. & Modarres, M. (2002). Local application of glyceril trinitrate ointment for primary dysmenorrhoea. International Journal of Gynaecology & Obstetrics, 79, 43-4.}. Local erythema is seen at the site of application in 10% of patients and slight drops in systolic and diastolic blood pressure are recorded, but no cases of hypotension or tachycardia have been noted {Ghazizadeh, S., Dadkhah, T. & Modarres, M. (2002). Local application of glyceril trinitrate ointment for primary dysmenorrhoea. International Journal of Gynaecology & Obstetrics, 79, 43-4.}. Although NO donors have been shown in vitro to inhibit spontaneous contractions of non-pregnant myometrium, the response is not mediated through the cGMP pathway, as soluble guanylate cyclase inhibitors fail to block the NO donor-mediated relaxation. Therefore it would seem unlikely that elevating cGMP levels (by administering a PDE inhibitor) would cause wide spread relaxation of the myometrium, and thus would not appear to provide a palpable approach to treating dysmenorrhoea. The response can however be blocked by calcium-dependent potassium channel blockers {Bradley, K. K., Buxton, I. L., Barber, J. E., McGaw, T. & Bradley, M. E. (1998). Nitric oxide relaxes human myometrium by a cGMP-independent mechanism. American Journal of Physiology, 275, C1668-73; Buxton, I. L., Kaiser, R. A., Malmquist, N. A. & Tichenor, S. (2001). NO-induced relaxation of labouring and non-labouring human myometrium is not mediated by cyclic GMP. British Journal of Pharmacology, 134, 206-14.}.

Although there is immunohistochemical data to show the presence of both eNOS and iNOS in human myometrium {Chwalisz, K. & Garfield, R. E. (2000). Role of nitric oxide in implantation and menstruation. [Review] Human Reproduction, 15, 96-111.}, in functional tissue experiments the relaxant effects of NO, on both spontaneous and agonist-induced contractility, has been shown to be through a cGMP-independent pathway {Bradley, K. K., Buxton, I. L., Barber, J. E., McGaw, T. & Bradley, M. E. (1998). Nitric oxide relaxes human myometrium by a cGMP-independent mechanism. American Journal of Physiology, 275, C1668-73; Buxton, I. L., Kaiser, R. A., Malmquist, N. A. & Tichenor, S. (2001). NO-induced relaxation of labouring and non-labouring human myometrium is not mediated by cyclic GMP. British Journal of Pharmacology, 134, 206-14.}. Therefore it would seem unlikely that elevating cGMP levels (by administering a PDE inhibitor) would cause wide spread relaxation of the myometrium, and thus would not appear to provide a palpable approach to treating dysmenorrhoea. Such a supposition is backed up by findings that, even at concentrations as high as 100 μM, PDE inhibitors (for example IBMX, zaprinast rolipram and Milrinone) have been shown to have variable suppressive activity (21-93%) of uterine contractions from myometrium taken from women under-going caesarean sections {Bardou, M., Cortijo, J., Loustalot, C., Taylor, S., Perales-Marin, A., Mercier, F. J., Dumas, M., Deneux-Tharaux, C., Frydman, R., Morcillo, E. J. & Advenier, C. (1999). Pharmacological and biochemical study on the effects of selective phosphodiesterase inhibitors on human term myometrium. Naunyn-Schmiedebergs Archives of Pharmacology, 360, 457-63.}.

We have hypothesised that the hypoxic pain associated with the reduction in blood flow to the myometrium may be a result of the direct effect of vaso-active agents (such as AVP) on the artery itself, as well as the contraction of the uterine smooth muscle, causing an occlusion of the myometrial blood vessels. Thus a reduction in the hypoxia, through increasing uterine blood flow (with a PDE inhibitor and/or V_{1A} antagonist), and a reduction in myometrial contractility (with a PDE inhibitor and/or V_{1A} antagonist) at the same time will result in a lower requirement for the doses of the combination of agents being used. In other words, an additional synergistic effect over and above that seen in the myometrium may be observed.

Surprisingly, it has now been found that combination therapy with an antagonist of the vasopressin receptor family and a PDE inhibitor results in unexpected and synergistic improvement in the treatment of dysmenorrhoea. When administered simultaneously, sequentially or separately, the vasopressin antagonist and PDE inhibitor may have the advantage that, due to a synergistic interaction between the active ingredients, they are more potent, have a longer duration of action, more effectively reduce disease progression and, therefore, the requirement for surgical intervention, have a broader range of activity, are more stable, have fewer side effects or are more selective (in particular they may have beneficial effects in dysmenorrhoea) or have other more useful properties than the compounds and combinations of the prior art.

The combination of the present invention not only provides a treatment of myometrial hypercontractility, uterine arterial vasoconstriction and subsequent pain, but also provides a treatment to reduce the basal tone of myometrium and uterine arteries, allowing them to remain in a more relaxed state. Although the dysmenorrhoea is cyclical, if the myometrium and uterine arteries maintain a more relaxed state each month then, in the long term, we hypothesis that this could lead to a reduction in the treatment required for future symptom relief.

Thus, in accordance with the invention, there is provided the use of a combination of (A) a PDE inhibitor or a pharmaceutically acceptable derivative thereof and (B) a vasopressin receptor antagonist or a pharmaceutically acceptable derivative thereof, in the manufacture of a medicament for the treatment of dysmenorrhoea.

Further there is provided a use of a combination of (A) and (B) as defined above for the treatment of dysmenorrhoea.

Still further, there is provided a use of a combination of (A) and (B) as defined above for the manufacture of a medicament for combination therapy by simultaneous, sequential or separate administration of (A) and (B) in the treatment of dysmenorrhoea.

Alternatively, there is provided a method of treating dysmenorrhoea comprising administering to a subject in need of such treatment amounts of (A) and (B) as defined above, which are together effective.

Further there is provided a pharmaceutical product containing (A) and (B) as defined above, as a combined preparation for simultaneous, separate or sequential use in treating dysmenorrhoea.

The dysmenorrhoea may be primary or secondary dysmenorrhoea. The secondary dysmenorrhoea may be a consequence of increased uterine tone, such as uterine fibroids or intra-uterine contraceptive devices.

In one aspect of the invention, the PDE target is selected from any one or more of the following PDE enzymes: PDE1, PDE2, PDE3, PDE4, PDE5, PDE7, PDE8, PDE9, PDE10, PDE11.

In one aspect of the invention, the vasopressin receptor antagonist inhibits a vasopressin receptor family member wherein the receptor is selected from any one or more of the following vasopressin receptor subtypes: V1a, V1b, V2 and Oxytocin. Preferably the receptor is selected from V1a or Oxytocin. More preferably the receptor is V1a.

Particular PDE enzymes of interest are as follows:

| Compound | Structure | Mode of action & References |
|---|---|---|
| Fia | 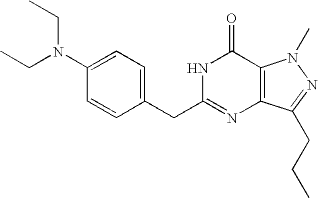 | I:PDE1 EP-A-0911333 (Example 50) |
| Fib | 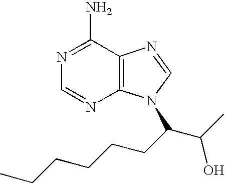 | I:PDE2 EHNA (an inhibitor of adenosinedeaminase) |
| FII | 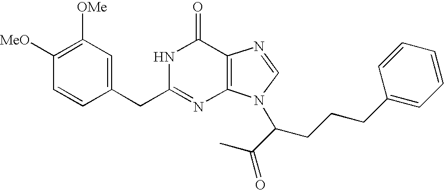 | I:PDE2 EP-A-0771799 (Example 100) |
| FIII | 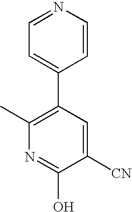 | I:PDE3 Milrinone (commercially available) |
| FIV | 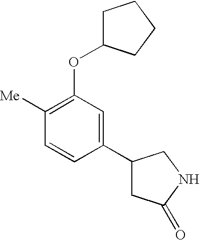 | I:PDE4 Rolipram (commercially available) |

Preferably the PDE inhibitor is a PDE4 or PDE5 inhibitor. More preferably the PDE inhibitor is a PDE5 inhibitor.

Inhibitors of the cGMP PDE5 enzyme (‘PDE5 inhibitors’) are characterized by compounds having high affinity and selectivity for the PDE5 enzyme, with little or no affinity for the other phosphodiesterase isoforms. They have been described for a number of indications. In particular, sildenafil: (5-[2-ethoxy-5-(4-methyl-1-piperazinylsulphonyl)phenyl]-1-methyl-3-n-propyl-1,6-dihydro-7H-pyrazolo[4,3-d]pyrimidin-7-one) (VIAGRA®) has been described for a number of cardiovascular disorders and has proved to be successful as the first orally effective treatment for male erectile dysfunction (MED).

The suitability of any particular PDE5 inhibitor can be readily determined by evaluation of its potency and selectivity using literature methods followed by evaluation of its toxicity, absorption, metabolism, pharmacokinetics, etc. in accordance with standard pharmaceutical practice.

Preferably the PDE5 inhibitors have an IC₅₀ at less than 100 nanomolar, more preferably, at less than 50 nanomolar, still more preferably at less than 10 nanomolar.

Preferably the PDE5 inhibitors used in the pharmaceutical combinations according to the present invention are selective for the PDE5 enzyme. Preferably they have a selectivity of PDE5 over PDE3 of greater than 100, more preferably greater than 300. More preferably, the PDE5 inhibitor has a selectivity over both PDE3 and PDE4 of greater than 100, more preferably, greater than 300. Selectivity ratios may be determined readily by the skilled person. IC₅₀ values for the PDE3 and PDE4 enzyme may be determined using established literature methodology, see S. A. Ballard et al, Journal of Urology, 1998, vol. 159, pages 2164-2171 and as detailed herein after.

Measurement of PDE5, PDE2, etc. inhibition is illustrated by the following assays.

Compounds suitable for use in accordance with the present invention are potent and selective PDE5 inhibitors . In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) can be obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors can be investigated by assaying a fixed amount of enzyme in the presence of varying inhibitor concentrations and low substrate, (cGMP or cAMP) in a 3:1 ratio unlabelled to [³H]-labeled at a conc ˜1/3 Kₘ) such that IC₅₀≅Kᵢ. The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates are re-sealed and shaken for 20 min, after which the beads are allowed to settle for 30 min in the dark and then counted on a TopCount plate reader (Packard, Meriden, Conn.). Radioactivity units are converted to % activity of an uninhibited control (100%), plotted against inhibitor concentration and inhibitor IC₅₀ values obtained using the ‘Fit Curve’ Microsoft Excel extension.

Functional Activity

This can be assessed in vitro by determining the capacity of a PDE5 inhibitor of the invention to enhance sodium nitroprusside or electric field stimulation-induced relaxation of pre-contracted rabbit corpus cavernosum tissue strips, as described by S. A. Ballard et al. (Brit. J. Pharmacol., 1996, 118 (suppl.), abstract 153P) or S. A. Ballard et al. (J. Urology, 1998, vol.159, 2164-2171).

In Vitro PDE Inhibitory Activities

In vitro PDE inhibitory activities against cyclic guanosine 3′,5′-monophosphate (cGMP) phosphodiesterases can be determined by measurement of their IC₅₀ values (the concentration of compound required for 50% inhibition of enzyme activity).

Suitable PDE5 inhibitors for the use according to the present invention may be any that satisfy the definition given above, and may include:
The PDE5 inhibitors mentioned in International Patent Application publication nos. WO03/000691; WO02/64590; WO02/28865; WO02/28859; WO02/38563; WO02/36593; WO02/28858; WO02/00657; WO02/00656; WO02/10166; WO02/00658; WO01/94347; WO01/94345; WO00/15639 and WO00/15228; and
U.S. Pat. Nos. 6,143,746; 6,143,747 and 6,043,252;
the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the hexahydropyrazino [2′,1′:6,1]pyrido [3,4-b]indole-1,4-diones disclosed in published international application WO95/19978; the pyrazolo [4,3-d]pyrimidin-4-ones disclosed in WO00/27848; the imidazo[5,1-f][1,2,4]triazin-ones disclosed in EP-A-1092719 and in published international application WO 99/24433 and the bicyclic compounds disclosed in published international application WO 93/07124; the pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719; the tricyclic compounds disclosed in EP-A-1241170; the alkyl sulphone compounds disclosed in published international application WO 02/074774; the compounds disclosed in published international application WO 02/072586; the compounds disclosed in published international application WO 02/079203 and the compounds disclosed in WO 02/074312.

The contents of the published patent applications and journal articles and in particular the general formulae of the therapeutically active compounds of the claims and exemplified compounds therein are incorporated herein in their entirety by reference thereto.

Preferred type V phosphodiesterase inhibitors (PDE5 inhibitors) for the use according to the present invention include:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
5-(2-ethoxy-5-morpholinoacetylphenyl)-1-methyl-3-n-propyl-1,6-dihydro-7H-pyrazolo[4,3-d]pyrimidin-7-one (see EP-A-0526004);
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);
(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);
5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);
(6R,12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351, Cialis®)), i.e. the compound of examples 78 and 95 of published international application WO95/19978, as well as the compound of examples 1, 3, 7 and 8;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA ®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433;
the compound of example 11 of published international application WO93/07124 (EISAI);
compounds 3 and 14 from Rotella D P, J. Med. Chem., 2000, 43,1257;
4-(4-chlorobenzyl)amino-6,7,8-trimethoxyquinazoline;
N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [“DA-8159” (Example 68 of WO00/27848)]; and
7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.

Still other type cGMP PDE5 inhibitors which may be useful in conjunction with the present invention include:4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

More preferably the PDE5 inhibitor is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one.

Most preferably the PDE5 inhibitor is sildenafil and pharmaceutically acceptable salts thereof. Sildenafil citrate is a preferred salt.

The vasopressin receptor family comprises V1a, V1b, V2 and Oxytocin receptors {Thibonnier M., Exp. Opin. Invest. Drugs (1998) 7(5), 729-740). The vasopressin receptor antagonist for use with the invention is preferably selective for the V1a receptor and the closely related oxytocin receptor. Activity at the oxytocin receptor may be beneficial. More preferably, the vasopressin receptor antagonist for use with the invention is selective for the V1a receptor.

Examples of vasopressin receptor antagonists, suitable for use in the present invention are disclosed in U.S. Pat. No. 6,090,818; EP0873309; WO 98/25901; WO 02/083685; JP 2000-63363; and WO 02/32864.

Examples of V1a receptor antagonists for use with the invention are: SR49049 (Relcovaptan), atosiban (Tractocile®)), conivaptan (YM-087) and OPC21268. Additionally, the V1a receptor antagonists described in WO 01/58880 are suitable for use in the invention.

Further examples of V1a antagonists for use with the invention are disclosed in PCT/IB03/04587 (unpublished) and GB application No. 0202852.8 (unpublished), in particular 8-chloro-5-Methyl-1-(3,4,5,6-tetrahydro-2H-[1,2′]bipyridinyl-4-yl)-5,6-dihydro-4H-2,3,5,10b-tetraazo-benzo[e]azulene, or a pharmaceutically acceptable salt or solvate thereof, is preferred.

GB application No. 0202852.8 discloses compounds of formula (I) 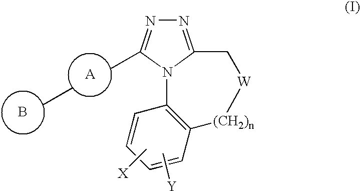
or pharmaceutically acceptable salts or solvates thereof, wherein,
W is O, S, or NR¹
R¹ represents H, C₁₋₆ alkyl, —(CH₂)ₐ—[C₃₋₈ cycloalkyl], phenyl, benzyl, pyridyl, pyrimidyl, —COR², —CO₂R², —CO—(CH₂)ₐ—NR²R³, —SO₂R², —(CH₂)_{b}—OR², —(CH₂)_{b}—NR²R³, or a saturated heterocycle of from 3 to 8 atoms containing one or more heteroatoms selected from O, N and S;
X and Y independently represent H, halogen, OH, CF₃, OCF₃, R⁴, —(CH₂)_{d}—CONR⁴R⁵, —(CH₂)_{d}—CN, —(CH₂)_{d}—SO₂NR⁴R⁵, —(CH₂)_{d}—NR⁴SO₂Me, —(CH₂)_{d}—COR⁴, —(CH₂)_{d}—OCOR⁴, —(CH₂)_{d}—NHCOR⁴, —(CH₂)_{d}—NR⁴COR⁵, —(CH₂)_{d}—OR⁶ or —(CH₂)_{d}—CO₂R⁶;
Ring A represents a piperidinyl, piperazinyl, pyrrolidinyl or azetidinyl group;
Ring B represents a phenyl, pyridinyl or pyrimidinyl group (optionally substituted with one or more groups independently selected from halogen, CN, CONH₂, CF₃, OCF₃, R⁷, and —(CH₂)_{f}—OR⁸);
R², R³, R⁴, R⁵and R⁷ independently represent H, straight or branched C₁₋₆ alkyl, —(CH₂)_{c}—[C₃₋₈ cycloalkyl], phenyl, benzyl, pyridyl or pyrimidyl;
or R² and R³, or R⁴ and R⁵, together with the nitrogen atom to which they are attached independently represent a heterocycle of from 3 to 8 atoms;
R⁶ and R⁸ independently represent H, straight or branched C₁₋₆ alkyl, —(CH₂)_{c}—[C₃₋₈ cycloalkyl], —(CH₂)ₑ—NR⁴R⁵, —(CH₂)ₑ—OR⁴, phenyl, benzyl, pyridyl or pyrimidyl;
n=0, 1 or 2;
a , c, d and f are all independently selected from 0, 1, 2 or 3;
b and e are independently selected from 2 or 3.

The following schemes illustrate the preparation of compounds of the formula (I), throughout which Rings A and B, and groups W, X, Y, and n are as defined above unless otherwise stated. (I′) represents (I) when W is NR¹.

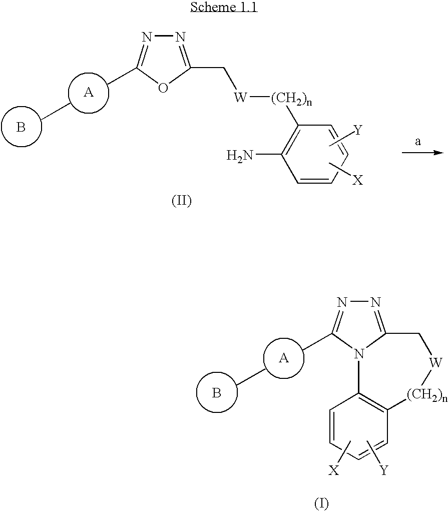

Step (a): Oxadiazole (II) is reacted with an acid catalyst to give the compound of formula (V). Typically the reaction is carried out by heating the starting materials to elevated temperatures, such as 100-150° C., for 1 to 48 hours with a suitable acidic catalyst such as p-TSA, or Lewis acid catalyst such as magnesium chloride, optionally using a high boiling solvent such as xylene.

Preferred conditions are:
Amine (II) and cat. P-TSA, in xylene at 140° C. for 48 hrs.

When W═NR¹, then:

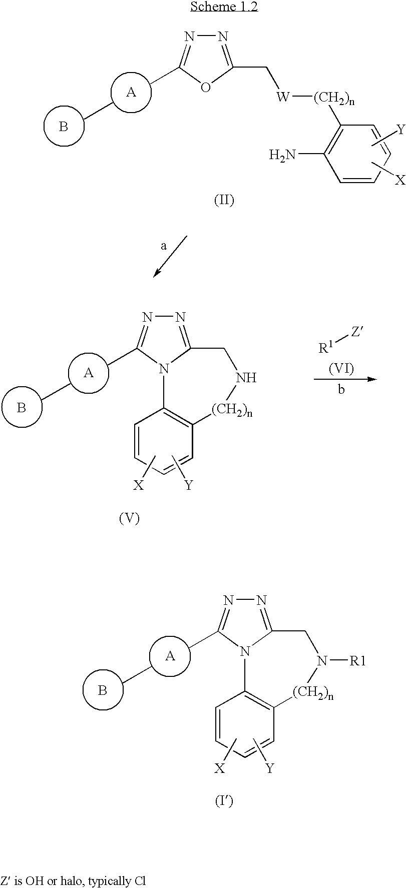

Compounds suitable for use as compound (VI) are commercially available or are known in the literature.

Step (b): The reaction of amine (V) with compound (VI) can be carried out by standard methods.

When R¹═COR², CO₂R², CO—(CH₂)_{b}—NR³R⁴, SO₂R² then, typically, the coupling may be undertaken by using:
(i) an acyl/sulphonyl/chloride (VI)+amine (V) with an excess of acid acceptor, in a suitable solvent; or
(ii) an acid (VI) with a conventional coupling agent+amine (V), optionally in the presence of a catalyst, with an excess of acid acceptor in a suitable solvent; and
(iii) when R¹ represents an Aryl group, an aryl halide (VI)+amine (V), optionally in the presence of a catalyst, with an excess of acid acceptor in a suitable solvent.

Typically the conditions are as follows:
Acylation/Sulphonylation, Z=Cl
(i) An excess of acyl/sulphonyl chloride (VI) (generated in-situ), 1 eq. of amine (V), optionally with an excess of 3° amine such as Et₃N, Hünig's base or NMM, in DCM or THF, without heating for 1 to 24 hrs.

The preferred conditions are:
Amine (V), 1.5 eq. acid/sulphonyl chloride (VI), 1.5 eq. NMM in DCM at rt. for 16 hours.
Amide Bond Formation. Z=OH
(ii) Excess acid (VI), WSCDI/DCC and HOBT/HOAT, 1 eq. of amine (V), with an excess of NMM, Et₃N, Hünig's base in THF, DCM or EtOAc, at rt. for 4 to 48 hrs; or
   excess acid (VI), PYBOP®/PyBrOP®/Mukaiyama's reagent, 1 eq. of amine (V), with an excess of NMM, Et₃N, Hünig's base in THF, DCM or EtOAc, at rt. for 4 to 24 hrs.
Arylation (R¹=Aryl, heteroaryl), Z=halo
(iii) Arylation of compound (V) can be carried out by a palladium catalysed cross-coupling reaction using a suitable base (t-BuONa), a catalytic amount of suitable additive such as 2,2′-bis(diphenylphosphino)-1,1′-binaphthyl and a suitable palladium catalyst in toluene at elevated temp for 1 to 24 hours under an inert atmosphere, to give compound (I′). Alternatively compound (I′) can be prepared by reaction of the amine (I) with compound (VI) by heating at elevated temperature, such as 50° C.-140° C., in a suitable solvent such as DMF, NMP or 1,4-dioxan for about 1-48 hrs with a base such as potassium carbonate, sodium hydrogen carbonate or Hünig's base.

Preferred conditions are:
1-2.5 eq. halide (VI), 1-2 eq. potassium carbonate in N,N-dimethylformamide at 50° C. for 4-18 hours; or
1-2.5 eq. halide (VI), 2-3 eq. Hünig's base, in 1,4-dioxan or NMP at reflux for 18-48 hrs; or
1 eq. Halide (VI), 3.5 eq. NaOt-Bu, 0.08eq BINAP, 0.4 eq. Pd(dba)₂, in toluene for 8 hrs at 70° C.

Alternatively, compounds (I′) may be prepared by the route shown below in Scheme 1.3.

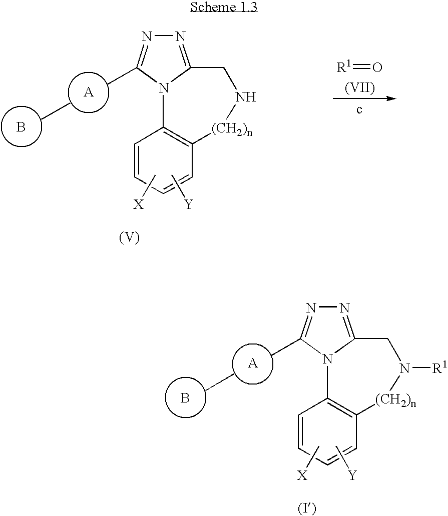

Compounds suitable for use as compound (VII) are commercially available or are known in the literature.

Step (c): Amine (V) is reacted with an excess of aldehyde/ketone (VIl) in the presence of a reducing agent, such as sodium triacetoxy borohydride or sodium cyanoborohydride, to give the compound of formula (I′). This reaction may be carried out by:
stirring the starting materials at temperatures such as 20° C.-80° C. for 1 to 48 hours in a suitable solvent such as dichloromethane, or
heating amine (V) with excess compound (VIl) with a suitable Lewis acid catalyst such titanium tetrachloride or titanium tetraisopropoxide at temperatures such as 50° C.-100° C. in a suitable solvent such as dichloroethane or ethanol for 1-18 hours, followed by reduction of the intermediate imine/iminium species with a suitable reducing agent, such as sodium borohydride, or hydrogenolysis over a suitable catalyst, such as platinum oxide or palladium on carbon.

Preferred conditions are:
Amine (V), 1.5 eq. Aldehyde/ketone (VII), 2.0 eq. sodium triacetoxy borohydride in dichloromethane at room temperature for 2 hours.

When ring B is linked to ring A via an N atom, and W represents O or S then:

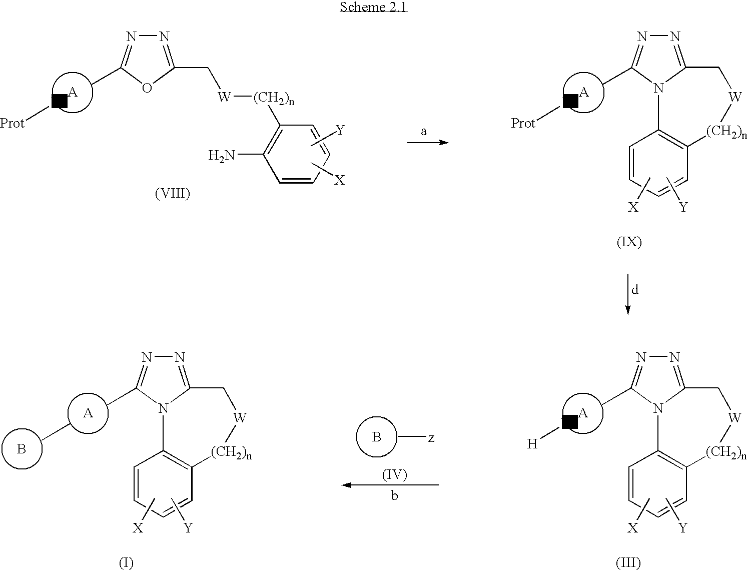

Prot represents a suitable protecting group for nitrogen, for example Boc, CBz or Allyl carbamate. Standard methodology for nitrogen protecting groups is used, such as that found in textbooks (e.g. “Protecting Groups in Organic Synthesis” by T. W. Greene and P. Wutz). Z represents a leaving group such as halogen.

Compounds suitable for use as compound (IV) are commercially available or are known in the literature.

Arylation of compound (III) can be carried out as described in Step (b) above.

Preferred conditions are:
1-2.5 eq. halide (IV), 1-2 eq. potassium carbonate in N,N-dimethylformamide at 50° C. for 4-18 hours; or
1-2.5 eq. halide (IV), 2-3 eq. Hünig's base, in 1,4-dioxan or NMP at reflux for 18-48 hrs; or
1 eq. halide (IV), 3.5 eq. NaOt-Bu, 0.08 eq BINAP, 0.4 eq. Pd(dba)₂, in toluene for 8 hrs at 70° C.

Step (d): Deprotection of compound (IX) is undertaken using standard methodology, as described in “Protecting Groups in Organic Synthesis” by T. W. Greene and P. Wutz”.

When Prot is Boc, the preferred methods are:
hydrogen chloride in a suitable solvent such as 1,4-dioxane at room temperature for 1-16 hours; or
a solution of trifluoroacetic acid in dichloromethane for 1-2 hours.

When Prot is CBz, the preferred method is hydrogenolysis using a suitable palladium catalyst in a solvent such as ethanol.

When Prot is an allyl carbamate, preferred conditions are thiobenzoic acid and a suitable palladium catalyst such as Pd₂(Dba)₃ with a suitable phosphine additive such as 1,4-bis(diphenylphosphino)butane in tetrahydrofuran for 20 minutes.

When ring B is linked to ring A via an N atom, and W represents NR¹ then:

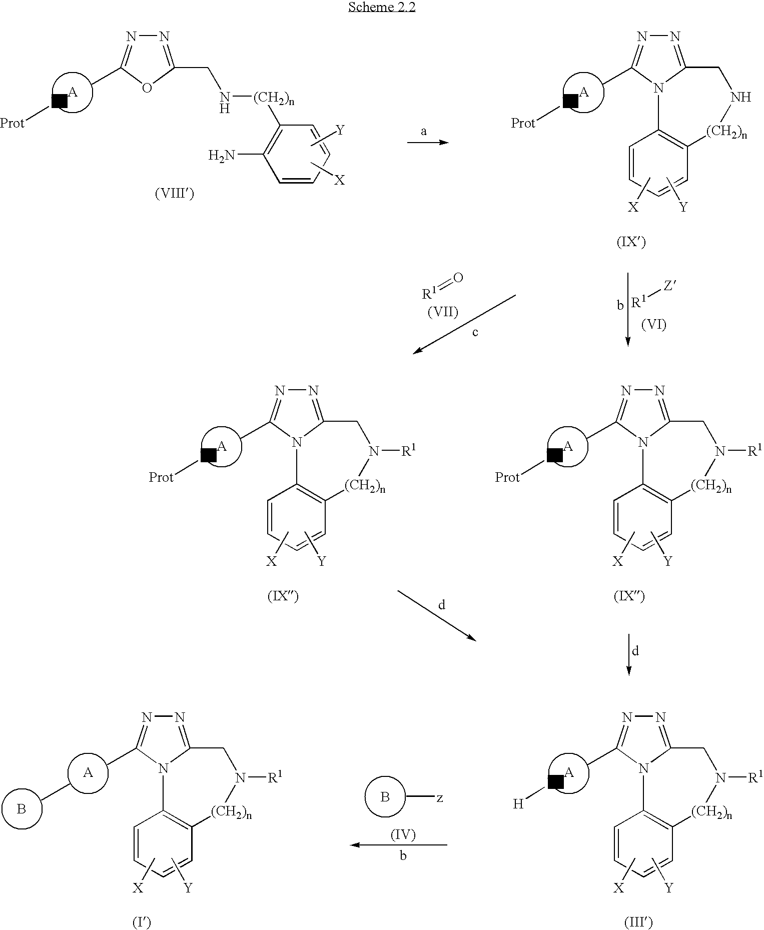

Prot represents a suitable protecting group for nitrogen, for example Boc, CBz or Allyl carbamate. Standard methodology for nitrogen protecting groups is used, such as that found in textbooks, (e.g. “Protecting Groups in Organic Synthesis” by T. W. Greene and P. Wutz).

Z represents halo (typically Cl). Z′ represents a leaving group (typically Cl or OH).

Compounds suitable for use as compound (IV) are commercially available or are known in the literature.

Compound (IX″) typically can be prepared from compound (IX′) using the methodology described in Step (b) and Step (c) above.

Compound (III′) typically can be prepared from compound (IX″) using the methodology described in Step (d) above.

Compounds (I′) typically can be prepared by arylation of compounds (III′) using the methodology described in Step (b) above.

Compounds suitable for use as compounds (II) and (VIII) are known in the literature or can be prepared as shown in schemes 3.1 and 3.2 below.

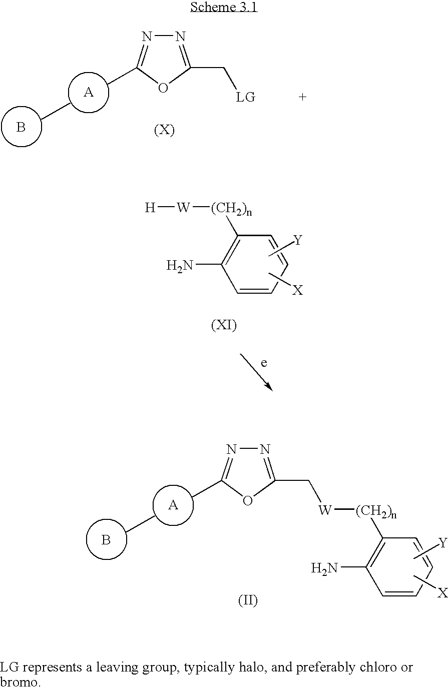

When rings A and B are linked through an N atom then:

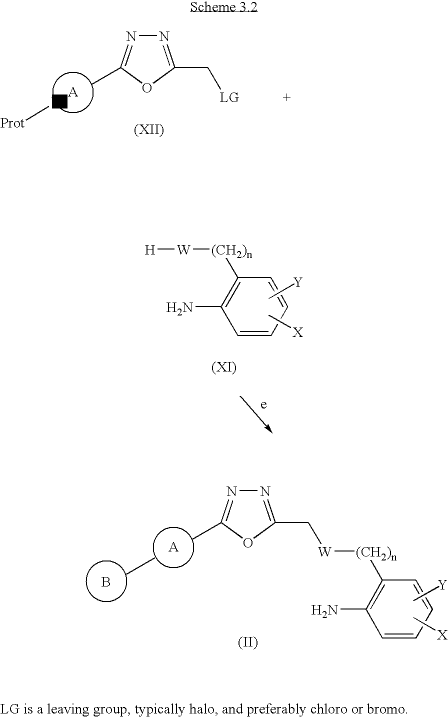

Compounds suitable for use as compounds (XI) are known in the literature or can be prepared using standard methodology: for example, reduction of benzoic acids or benzonitriles.

When W represents NR¹:

Step (e): Compound (X)/(XII) is reacted with an excess of compound (XI) to give compound (II)/(VIII) respectively, optionally in the presence of an excess of base, such as triethylamine, Hünig's base or potassium carbonate as proton acceptor, in a suitable high boiling solvent such as THF, Toluene or DMF at temperatures from 50° C. to 100° C. for 1 to 48 hours.

Preferred conditions are:
2.5 eq. of compound (XI) in THF at 50° C. for 48 hours.

When W represents O or S:

Step (e): Compound (X)/(XII) is reacted with an excess of compound (XI) in the presence of a base such as sodium hydride, potassium hexamethyldisilazide, ⁿbutyl lithium or isopropyl magnesium chloride, in a suitable solvent such as THF, Toluene or NMP at temperatures from 0° C. to 50° C. for 1 to 24 hours, to give compound (II)/(VIII) respectively.

Preferred conditions are:
3 eq. of compound (XI) and 2.5 eq. of NaH in THF at 20° C. for 2 hours. Compounds suitable for use as compounds (X) and (XII) are known in the literature or can be prepared as shown in scheme 4.1 and 4.2.

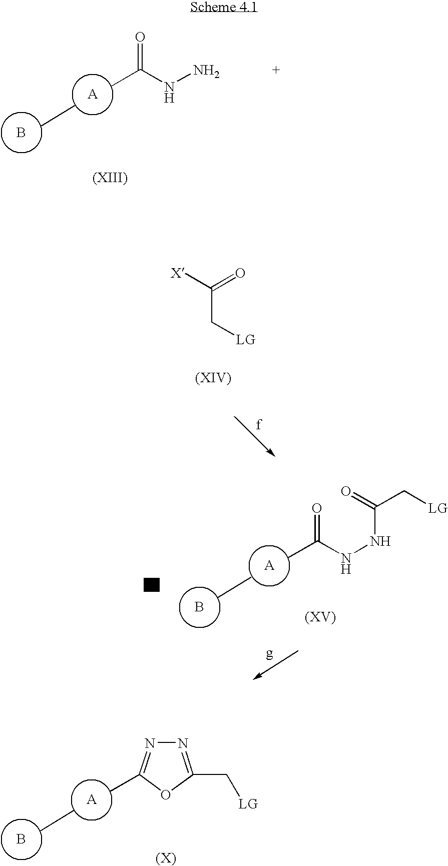

X′ represents OH or halo, and preferably represents Cl. LG represents a leaving group, typically halo, and preferably chloro or bromo

When rings A and B are linked through an N atom then: 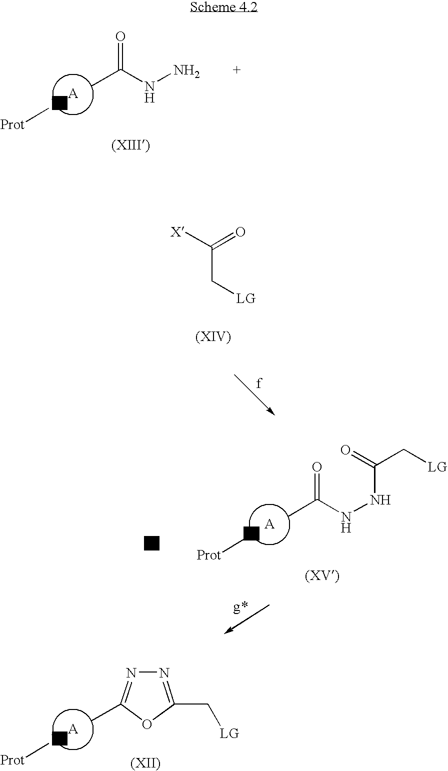
X′ represents OH or halo, and preferably represents Cl. LG is a leaving group, typically halo, and preferably chloro or bromo

Compound (XIV) is either commercially available or is known in the literature.

Step (f): The reaction of hydrazide (XIII/XIII′) with compound (XIV) can be carried out by standard methods.

Coupling may be undertaken by using either:
(i) an acyl chloride (XIV)+hydrazide (XIII/XIII′) with an excess of acid acceptor in a suitable solvent; or
(ii) acid (XIV) with a conventional coupling agent+hydrazide (XIII/XIII′), optionally in the presence of a catalyst, with an excess of acid acceptor in a suitable solvent.

Typically the conditions are as follows:
(i) acid chloride (XIV) (generated in-situ), an excess of hydrazide (XIII/XIII′) optionally with an excess of 3° amine such as Et₃N, Hünig's base or NMM, in DCM or THF, without heating for 1 to 24 hrs; or
(ii) acid (XIV), WSCDI/DCC and HOBT/HOAT, an excess of hydrazide (XIII/XIII′), with an excess of NMM, Et₃N, Hünig's base in THF, DCM or EtOAc, at rt. for 4 to 48 hrs; or
(ii) acid (XIV), PYBOP®/PyBrOP®/Mukaiyama's reagent, an excess of hydrazide (XIII/XIII′), with an excess of NMM, Et₃N, Hünig's base in THF, DCM or EtOAc, at rt. for 4 to 24 hrs.

The preferred conditions are:
Hydrazide (XIII/XIII′), 1.5 eq. chloro acetyl chloride (XIV), 1.5 eq. NMM in DCM at rt. for 16 hours.

Step (g): Cyclisation of compound (XV/XV′) is carried out under suitable dehydrating conditions, at elevated temperatures for up to 18 hours.

Typically, dehydrating agents such as polyphosphoric acid, phosphorous oxychloride, triflic anhydride are used at temperatures from 20 to 120° C. for 5 minutes to 12 hours. Optionally, the reaction can be carried out in the presence of a base such as pyridine and suitable solvents such as dichloromethane and acetonitrile. Alternatively, the oxadiazole (XII/X) may be prepared according to the method of Rigo et. al. Synth. Commun. 16(13),1665, 1986.

Preferred conditions are:
Phosphorous oxychloride at 100° C. for 8 hours, or 2.5 eq. triflic anhydride, 5 eq. pyridine in dichloromethane at 20° C. for 3 hours.

Compounds suitable for use as compounds (XIII/XIII′) are known in the literature or can be prepared as shown in scheme 5.1 and 5.2.

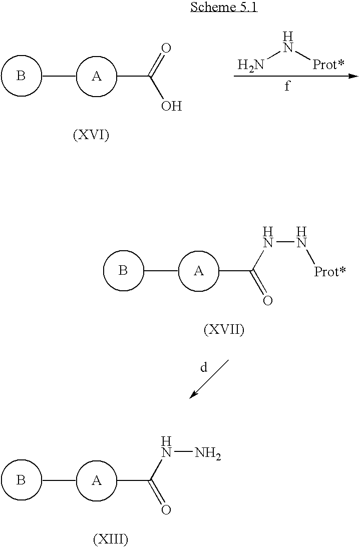

When rings A and B are linked through an N atom then:

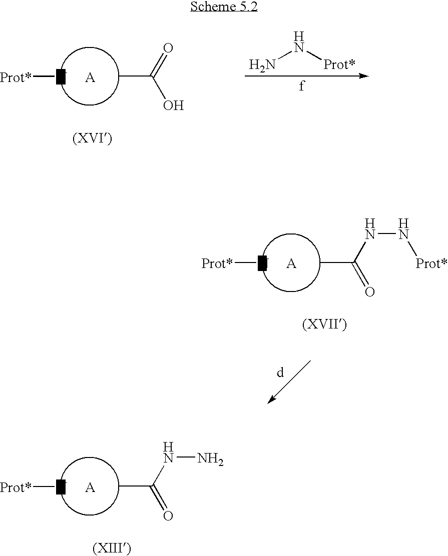

Compounds (XVI)/(XVI′) and protected hydrazine are either commercially available or are known in standard methodology such as the hydrolysis of the corresponding ester.

Carboxylic acid (XVI)/(XVI′) and protected hydrazine, where prot* is typically Boc, may be coupled to give compound (XVII/XVII′) respectively, using the conditions described above for the preparation of (XV/XV′), and then prot* is removed using standard methodology as described in Step (d) as described above, to give (XIII/XIII′).

Alternative routes to compound (XIII/XIII′) are shown below in schemes 6.1 and 6.2:

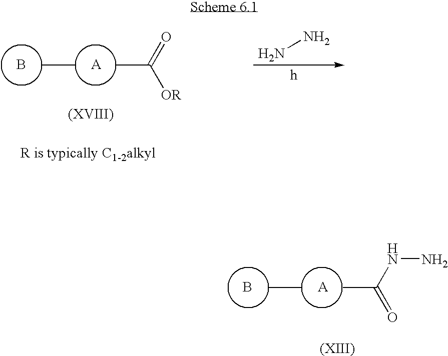

When rings A and B are linked through an N atom then:

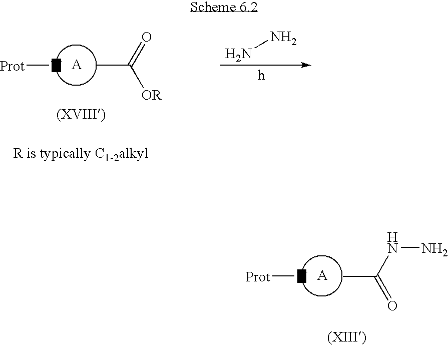

Step (h): The ester (XVIII/XVIII′) may be reacted with hydrazine in a suitable solvent, such as methanol, at an elevated temperature to provide the hydrazide (XVII/XVII′).

Preferred conditions:
3 eq. hydrazine, in methanol, at reflux for 18 hrs.

The vasopressin receptor antagonists for use in the invention may be tested in the screens set out below:

1.0 V_{1A} Filter Binding Assay

1.1 Membrane Preparation

Receptor binding assays were performed on cellular membranes prepared from CHO cells stably expressing the human V_{1A} receptor, (CHO-hV_{1A}). The CHO-hV_{1A} cell line was kindly provided under a licensing agreement by Marc Thibonnier, Dept. of Medicine, Case Western Reserve University School of Medicine, Cleveland, Ohio. CHO-hV_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. For bulk production of cell pellets, adherent CHO-hV_{1A} cells were grown to confluency of 90-100% in 850 cm² roller bottles containing a medium of DMEM/Hams F12 Nutrient Mix supplemented with 10% fetal bovine serum, 2 mM L-glutamine and 15 mM HEPES. Confluent CHO-hV_{1A} cells were washed with phosphate-buffered saline (PBS), harvested into ice cold PBS and centrifuged at 1,000 rpm. Cell pellets were stored at −80° C. until use. Cell pellets were thawed on ice and homogenised in membrane preparation buffer consisting of 50 mM Tris-HCl, pH 7.4, 5 mM MgCl₂ and supplemented with a protease inhibitor cocktail, (Roche). The cell homogenate was centrifuged at 1000 rpm, 10 min, 4° C. and the supernatant was removed and stored on ice. The remaining pellet was homogenised and centrifuged as before. The supernatants were pooled and centrifuged at 25,000×g for 30 min at 4° C. The pellet was resuspended in freezing buffer consisting of 50 mM Tris-HCl, pH 7.4, 5 mM MgCl₂ and 20 % glycerol and stored in small aliquots at −80° C. until use. Protein concentration was determined using Bradford reagent and BSA as a standard.

1.2 V_{1A} Filter Binding

Protein linearity followed by saturation binding studies were performed on each new batch of membrane. Membrane concentration was chosen that gave specific binding on the linear portion of the curve. Saturation binding studies were then performed using various concentrations of [³H]-arginine vasopressin, [³H]-AVP (0.05 nM-100 nM) and the K_{d} and Bₘₐₓ determined.

Compounds were tested for their effects on [³H]-AVP binding to CHO-hV_{1A} membranes, (³H-AVP; specific activity 65.5 Ci/mmol; NEN Life Sciences). Compounds were solubilised in dimethylsulfoxide (DMSO) and diluted to working concentration of 10% DMSO with assay buffer containing 50 mM Tris-HCl pH 7.4, 5 mM MgCl₂ and 0.05% BSA. 25 μl compound and 25 μl [³H]-AVP, (final concentration at or below K_{d} determined for membrane batch, typically 0.5 nM-0.6 nM) were added to a 96-well round bottom polypropylene plate. The binding reaction was initiated by the addition of 200 μl membrane and the plates were gently shaken for 60 min at room temperature. The reaction was terminated by rapid filtration using a Filtermate Cell Harvester (Packard Instruments) through a 96-well GF/B UniFilter Plate which had been presoaked in 0.5% polyethyleneimine to prevent peptide sticking. The filters were washed three times with 1 ml ice cold wash buffer containing 50 mM Tris-HCl pH 7.4 and 5 mM MgCl₂. The plates were dried and 50 μl Microscint-0 (Packard instruments) was added to each well. The plates were sealed and counted on a TopCount Microplate Scintillation Counter (Packard Instruments). Non-specific binding (NSB) was determined using 1 μM unlabelled d(CH2)5Tyr(Me)AVP ([β-mercapto-β,β-cyclopentamethylenepropionyl, 0-Me-Tyr²,Arg⁸]-vasopressin ) (βMCPVP), (Sigma). The radioligand binding data was analysed using a four parameter logistic equation with the min forced to 0%. The slope was free fitted and fell between −0.75 and −1.25 for valid curves. Specific binding was calculated by subtracting the mean NSB cpm from the mean Total cpm. For test compounds the amount of ligand bound to the receptor was expressed as % bound=(sample cpm−mean NSB cpm)/specific binding cpm×100. The % bound was plotted against the concentration of test compound and a sigmoidal curve was fitted. The inhibitory dissociation constant (Kᵢ) was calculated using the Cheng-Prusoff equation: Kᵢ=IC₅₀/(1+[L]/K_{d}) where [L] is the concentration of ligand present in the well and K_{d} is the dissociation constant of the radioligand obtained from Scatchard plot analysis.

2.0 V_{1A} Functional Assay; Inhibition of AVP/V_{1A}-R Mediated Ca²⁺ Mobilization by FLIPR (Fluorescent Imaging Plate Reader) (Molecular Devices)

Intracellular calcium release was measured in CHO-hV_{1A} cells using FLIPR, which allows the rapid detection of calcium following receptor activation. The CHO-hV_{1A} cell line was kindly provided under a licensing agreement by Marc Thibonnier, Dept. of Medicine, Case Western Reserve University School of Medicine, Cleveland, Ohio. CHO-V_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. On the afternoon before the assay cells were plated at a density of 20,000 cells per well into black sterile 96-well plates with clear bottoms to allow cell inspection and fluorescence measurements from the bottom of each well. Wash buffer containing Dulbecco's phosphate buffered saline (DPBS) and 2.5 mM probenecid and loading dye consisting of cell culture medium containing 4 μM Fluo-3-AM (dissolved in DMSO and pluronic acid),(Molecular Probes) and 2.5 mM probenecid was prepared fresh on the day of assay. Compounds were solubilised in DMSO and diluted in assay buffer consisting of DPBS containing 1% DMSO, 0.1% BSA and 2.5 mM probenecid. The cells were incubated with 100 μl loading dye per well for 1 hour at 37° C. in humidified atmosphere with 5% CO₂. After dye loading the cells were washed three times in 100 μl wash buffer using a Denley plate washer. 100 μl wash buffer was left in each well. Intracellular fluorescence was measured using FLIPR. Fluorescence readings were obtained at 2 s intervals with 50 μl of the test compound added after 30 s. An additional 155 measurements at 2 s intervals were then taken to detect any compound agonistic activity. 50 μl of arginine vasopressin (AVP) was then added so that the final assay volume was 200 μl. Further fluorescence readings were collected at 1 s intervals for 120 s. Responses were measured as peak fluorescence intensity (FI). For pharmacological characterization a basal FI was subtracted from each fluorescence response. For AVP dose response curves, each response was expressed as a % of the response to the highest concentration of AVP in that row. For IC₅₀ determinations, each response was expressed as a % of the response to AVP. IC50 values were converted to a modified K_{b} value using the Cheng-Prusoff equation which takes into account the agonist concentration, [A], the agonist EC₅₀ and the slope: K_{b}=IC₅₀/(2+[A]/A₅₀]ⁿ)^{1/n}−1 where [A] is the concentration of AVP, A₅₀ is the EC₅₀ of AVP from the dose response curve and n=slope of the AVP dose response curve.

The compounds for use in the present combination invention can exist in unsolvated forms as well as solvated forms, including hydrated forms. In general, the solvated forms, including hydrated forms, which may contain isotopic substitutions (e.g. D2O, d6-acetone, d6-DMSO), are equivalent to unsolvated forms and are encompassed within the scope of the present invention.

The compounds for use in the present invention possess may one or more chiral centers and each center may exist in the R(D) or S(L) configuration. The present invention includes all enantiomeric and epimeric forms as well as the appropriate mixtures thereof. Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof.

Also included within the present scope of the compounds for use in the invention are polymorphs thereof.

In a further embodiment there is provided a pharmaceutical composition comprising a mixture of effective amounts of (A) as hereinbefore defined and (B) as hereinbefore defined, optionally together with a pharmaceutically acceptable carrier, for administration either prophylactically or when pain commences.

In the pharmaceutical compositions of the present invention, (A) is present in an amount of from 1 mg up to 1000 mg per dose, and (B) is present in an amount of from 1 mg up to 1000 mg per dose. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The pharmaceutical compositions of the present invention can be administered alone but will generally be administered as a formulation in association with one or more pharmaceutically acceptable excipients. The term “excipient” is used herein to describe any ingredient other than the compound of the invention. The choice of excipient will to a large extent depend on the particular mode of administration.

The compounds for use in the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example water, ethanol, propylene glycol, methylcellulose, or a suitable oil, and one or more emulsifying agents and/or suspending agents. Liquid formulations may also be prepared by the reconstitution of a solid, for example, from a sachet.

The compounds for use in the invention may also be used in fast-dissolving, fast disintegrating dosage forms such as those described in Expert Opinion in Therapeutic Patents, 11(6), 981-986 by Liang and Chen (2001).

A typical tablet may be prepared using standard processes known to a formulation chemist, for example, by direct compression, granulation (dry, wet or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose (HPC), hydroxypropylmethylcellulose (HPMC) and gelatin, disintegrants, for example, sodium starch glycollate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

The compounds for use in the invention may also be administered directly into the blood stream, into muscle, or into an internal organ. Suitable means for parenteral administration include intravenous, intraarterial, intreperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free, water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

The compounds for use in the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J. Pharm. Sci., 88(10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds for use in the invention may be formulated in a more solid form for administration as an implanted depot providing long-term release of the active compound.

The compounds for use in the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitans trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 1 μl to 1001 μl. A typical formulation may comprise a compound of formula (I), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol, or magnesium stearate.

Formulations for inhaled/intranasal administration may be formulated to be immediate and/or modified release. Modified release formulations include delayed-, sustained-, pulsed-, controlled dual-, targeted and programmed release.

The compounds for use in the invention may be administered rectally, vaginally or via the intrauterine route, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

The compounds for use in the invention may also be administered directly to the eye or ear, typically in the form of drugs of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

The compounds for use in the invention may be combined with soluble macromolecular entities such as cyclodextrin or polyethyleneglycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

The compositions of the present invention may be administered by direct injection. For some applications, preferably the agent is administered orally. For some applications, preferably the agent is administered topically.

Pharmaceutical compositions according to the invention may contain 0.1%-95% of the compounds of this invention, preferably 1%-70%.

“Effective amounts” as used herein is an amount of (A) and (B) that will elicit the biological or medical response being sought. The daily dose of (A) and (B) employed in the method of treatment is similar to the doses described for use in the pharmaceutical compositions hereinbefore described. In the method of treatment according to the present invention (A) and (B) can be administered together combined in a single dosage form, or they can be administered separately, essentially concurrently, each in its own dosage form but as part of the same therapeutic treatment program, and it is envisaged that (A) and (B) may be separately administered, at different times and by different routes.

### EXAMPLES

Myometrial Synergy Studies

Methodology:

The effect of the V_{1A} antagonist/PDE inhibitor combination treatment on myometrial contractility can be investigated using in vitro functional organ bath studies. Uterine smooth muscle, obtained from women undergoing hysterectomies (or any human or animal smooth muscle preparation containing V_{1A} receptors), for example, uterine smooth muscle, obtained from women undergoing hysterectomies, is tensioned to 1 g in Krebs buffer at 37° C. (note that some studies may need to be conducted under different experimental conditions, e.g. at 32° C. or different parameters, with reduced calcium, in order to reduce the spontaneous contractility of the tissue). A contractile response to KCl is obtained in each tissue to check its viability and future data can be expressed as a percentage of this contractile response. Two types of experiments can then conducted:
1. A PDE inhibitor (e.g. 3-ethyl-5-{5-[(4-ethylpiperazino)sulphonyl]-2-propoxyphenyl}-2-(2-pyridylmethyl)-6,7-dihydro-2H-pyrazolo[4,3-d]pyrimidin-7-one) is administered either alone or in combination with a V_{1A} antagonist (e.g. SR49059) and the effects on basal tone examined. Subsequently a concentration dependent dose response curve to arginine vasopressin (AVP) is conducted. Any changes in basal tone are taken into account when analysing the subsequent AVP dose response curve. Appropriate controls are included for each part of the experiment.
2. Tissues are contracted with AVP. On reaching a steady contractile response either, a single dose or, a concentration response curve to a PDE inhibitor, V_{1A} antagonist or combination of the two can be administered. Appropriate controls are included for each part of the experiment.
   Results of Myometrial Synergy Studies

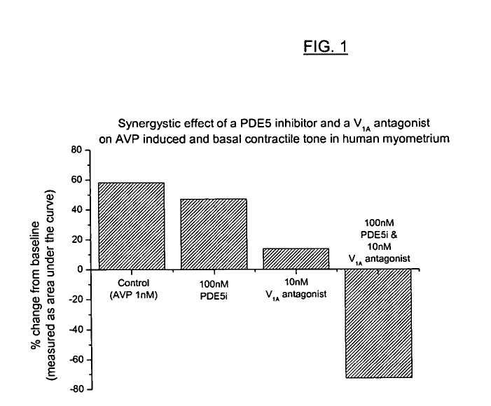

Human myometrium has an intrinsic contractile activity. In the presence of AVP the number of inherent uterine contractions is increased significantly in patients (Akerlund, 1997). When AVP is administered ex-vivo onto uterine smooth muscle strips an increase in uterine contractions is also seen (as measured by area under the curve). In the presence of 100 nM of a PDE5 inhibitor there is no significant change in the number of AVP induced contractions, but in the presence of a V_{1A} antagonist there is a 76% reduction in the AUC (an effect also seen in patients (Bossmar et al 1997)). As shown in FIG. 1, when the PDE5i and V_{1A} antagonist are administered in combination there is a marked synergistic effect on inhibiting uterine contractility. Not only is all the agonist (AVP) induced contractile activity inhibited, but basal myometrial contractile tone is also reduced. Therefore, as demonstrated in FIG. 1, a combination of a V_{1A} antagonist and a PDE inhibitor not only effectively suppresses myometrial hyper-contractility, as seen in dysmenorrhoeic women, but also unexpectedly decreases the basal myometrial tone. Thus resulting in long term beneficial effects slowing disease progression.

Through the synergistic action exhibited by the combination, such a therapy opens up the possibility of increased efficacy, including increased efficacy in the most severe cases, in addition to the possibility that the doses of each individual agent required to have an effect may be reduced and hence decreases the chance of any side effects. Furthermore, if the myometrium and uterine arteries maintain their relaxed state then reduction in the duration of treatment required may occur.

Uterine Artery Synergy Studies

Methodology for Artery Synergy Studies:

Pre-clinically the efficacy of the PDE5i on increasing blood flow and the V_{1A} antagonist on reducing uterine smooth muscle contractility can be shown using in vitro functional organ bath studies.
1. Uterine artery from women undergoing hysterectomy (or any human or animal arterial preparation containing V_{1A} receptors) is tensioned to 2 g initially and then readjusted to 1 g in Krebs buffer at 37° C. The tissue is contracted to phenylephrine and a concentration dose response curve to acetylcholine obtained. If the artery relaxes by >60% the endothelium of the artery is deemed to be intact. The arterial rings with an intact endothelium are re-contracted to phenylephrine and a concentration response curve to the PDE inhibitor obtained.
2. Uterine smooth muscle, obtained from women undergoing hysterectomies (or any human or animal smooth muscle preparation containing V_{1A} receptors), is tensioned to 1 g in Krebs buffer (with reduced calcium) at 32° C. (the reduced temperature and calcium are required to dampen the spontaneous contractility of the tissue). A contractile response to KCl is obtained in each tissue to check its viability and future data can be expressed as a percentage of this contractile response. The V_{1A} antagonist is administered prior to obtaining a concentration response curve to AVP. Alternatively the methodology for the myometrial synergy studies can be used.
   Clinical Studies to Investigate Synergy in the Myometrium and Uterine Arteries:

Both the individual components and the combination therapy are tested clinically using oral therapies in women suffering from primary dysmenorrhoea. In a randomised, double blind placebo controlled study, utilising 12 women with a history of primary dysmenorrhoea, the effects of a PDE inhibitor or V_{1A} antagonist (e.g. SR49059) on their own or in combination are examined. Experiments must be performed on three occasions within the first three days of three, usually consecutive, menstrual cycles. Uterine artery blood flow is measured using either 3-D Doppler velocimetry, 2-D colour Doppler (measured as the power Doppler signal intensity) or contrast enhanced MRI and uterine smooth muscle contractility by either the implantation of intrauterine uterine pressure catheters (measured as area under the intrauterine pressure curve (AUC)), 3-D ultrasonography or ischaemic biomarkers. Both uterine blood flow and myometrial contractility are studied at time intervals before and after drug administration to the patients. Lower abdominal pain can also be continuously recorded on a 10 cm visual analogue scale (VAS) graded from “no pain” to “maximal pain.

### Synthesis of 8-chloro-5-Methyl-1-(3,4,5,6-tetrahydro-2H-[1,2′]bipyridinyl-4-yl)-5,6-dihydro-4H-2,3,5,10b-tetraazo-benzo[e]azulene

### Preparation 1: 3,4,5,6-Tetrahydro-2H-[1,2′]bipyridinyl-4-carboxylic acid hydrazide

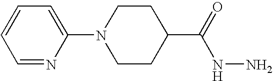

3,4,5,6-Tetrahydro-2H-[1,2′]bipyridinyl-4-carboxylic acid ethyl ester (1 g, 4.3 mmol)(see Farmaco, 1993, 48(10), 1439) was dissolved in methanol (20 ml) containing hydrazine hydrate (620 μl, 20 mmol) and was heated under reflux for 18 hours. The mixture was cooled to room temperature and evaporated under reduced pressure. The solid formed was triturated with propan-2-ol to give the title compound as a white solid (493 mg).

APCI MS m/z 221. [M+H]⁺

### Preparation 2: 3,4,5,6-Tetrahydro-2H-[1,2′]bipyridinyl-4-carboxylic acid N′-(2-chloro-acetyl)-hydrazide

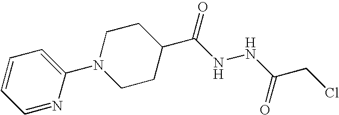

The hydrazide of Preparation 1 (23.6 g, 0.11 mol) was suspended in dichloromethane (500 ml) and 4-methylmorpholine (17.7 ml, 0.16 mol) was added. The mixture was cooled using an ice bath and chloroacetyl chloride (12.8 ml, 0.16 mol) was added dropwise. The reaction was warmed to room temperature and was stirred for 3 hours. The solid formed was isolated by filtration, washed with dichloromethane and diethyl ether, and dried under vacuum to give the title compound (20.4 g).

LCMS: m/z ES+ 297 [M+H]⁺

### Preparation 3: 4-(5-Chloromethyl-[1,3,4]oxadiazol-2-yl)-3,4,5,6-tetrahydro-2H-[1,2′]bipyridinyl

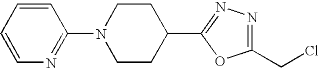

The hydrazide of Preparation 2 (20.4 g, 69 mmol) was suspended in phosphorus oxychloride (150 ml) at 100° C. for 4 hours. The mixture was cooled and the solvent was evaporated under reduced pressure. The residue was dissolved in ethyl acetate and was added to water. The aqueous layer was basified by addition of solid sodium hydrogen carbonate and the phases were separated. The aqueous phase was extracted with ethyl acetate (×2) and the combined organic layers were dried over magnesium sulphate and evaporated under reduced pressure. The material isolated was triturated with diethyl ether to give the title compound as a beige solid (15 g).

¹H NMR (400 MHz, CD₃OD): δ 1.91 (m, 2H), 2.19 (m, 2H), 3.14 (m, 2H), 3.30 (m, 1H), 4.29 (m, 2H), 4.86 (s, 2H), 6.69 (m,1H), 6.89 (d,1H), 7.58 (m, 1H), 8.08 (d, 1H)

### Preparation 4: 2-Aminomethyl-4-chloro-phenylamine

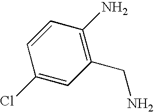

2-Amino-5-chloro-benzonitrile (9.0 g, 59 mmol) in tetrahydrofuran (100 ml) was added dropwise to an ice cooled 1 molar solution of lithium aluminium hydride (100 ml) in tetrahydrofuran and the reaction mixture was stirred at room temperature for 18 hours. Water (10 ml) was added dropwise. The resulting emulsion was dried over magnesium sulphate, filtered and evaporated under reduced pressure to give the title compound as a white solid (4.56 g).

¹H NMR (400 MHz, CDCl₃): δ 3.85 (s, 2H), 4.50 (s, 2H), 6.60 (d,1H), 7.05 (m, 2H)

### Preparation 5: 4-Chloro-2-({[5-(3,4,5,6-tetrahydro-2H-[1,2′]bipyridinyl-4-yl)-[1,3,4]oxadiazol-2-ylmethyl]-amino}-methyl)-phenylamine

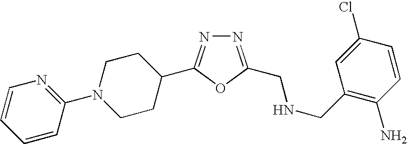

A solution of the amine of preparation 4 (6.4 g, 41 mmol) in tetrahydrofuran (50 ml) was added to a solution of the oxadiazole of preparation 3 (4.56 g, 16 mmol) in tetrahydrofuran (50 ml) and the mixture was heated to 50° C. for 18 hours. The reaction mixture was evaporated under reduced pressure and the residue was purified by chromatography on silica gel using methanol in dichloromethane as eluant (5:95), to give the title compound as a white solid (4.65 g).

APCl MS m/z 399 [MH]⁺

¹H NMR (400 MHz, CDCl₃): δ 1.95 (m, 2H), 2.20(m, 2H), 3.10 (m, 2H), 3.20 (m, 1H), 3.80(s, 2H), 4.00 (s, 2H), 4.30 (m, 2H), 6.60 (m, 1H), 6.65 (t, 1H), 6.70 (d, 1H), 7.00 (s, 1H), 7.05 (d,1H), 7.50 (t, 1H), 8.20 (d,1H)

### Preparation 6: 8-Chloro-1-(3,4,5,6-tetrahydro-2H-[1,2′]bipyridinyl-4-yl)-5,6-dihydro-4H-2,3,5,10b-tetraaza-benzo[e]azulene

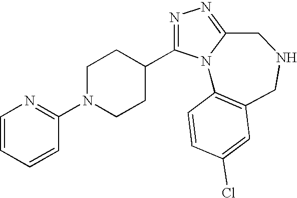

Toluene-4-sulfonic acid (100 mg, 0.58 mmol) was added to a solution of the oxadiazole of preparation 5 (4.65 g, 12 mmol) and heated to 140° C. for 18 hours. The mixture was cooled and purified by chromatography on silica gel using methanol and ammonium hydroxide in dichloromethane (5:0.5:95) as eluant to give the title compound (2.0 g) as an off-white solid.

APCl MS m/z 381 [MH]⁺, 403 [MNa]⁺

¹H NMR (400 MHz, CDCl₃): δ 1.80-2.20 (m, 4H), 2.95 (m, 2H), 3.14 (m, 1H), 3.68 (s, 2H), 3.92 (s, 2H), 4.36 (m, 2H), 6.60 (m, 1H), 6.67 (d, 1H), 7.35 (d, 1H), 7.50 (m, 3H), 8.17 (d, 1H)

Found; C, 59.90; H, 5.48; N, 20.50; C₂₀H₂₁N₆Cl 0.33CH₂Cl₂ requires; C, 59.72; H, 5.34; N, 20.55%.

### Example 1

### 8-Chloro-5-methyl-1-(3,4,5,6-tetrahydro-2H-[1,2′]bipyridinyl-4-yl)-5,6-dihydro-4H-2,3,5,10b-tetraaza-benzo[e]azulene trihydrochloride

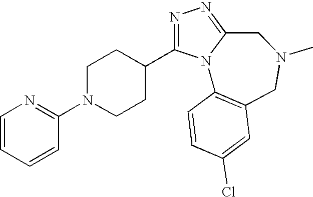

Formaldehyde (37% w/v aqueous, 0.1 ml, 1.2 mmol) was added to a solution of the amine of preparation 6 (200 mg, 0.53 mmol) in dichloromethane (5 ml). The mixture was stirred at room temperature for 0.25 hours before sodium triacetoxyborohydride (500 mg, 2.4 mmol) was added, and the reaction mixture was stirred for a further 18 hours. The reaction mixture was partitioned between 2N aqueous sodium hydroxide solution (10 ml) and dichloromethane (10 ml). The organic layer was evaporated under reduced pressure and purified by chromatography on silica gel using methanol in dichloromethane (5:95) as eluant. The residue was dissolved in dichloromethane (2 ml) and hydrochloric acid (1M in diethyl ether, 2 ml) was added. The solvent was evaporated under reduced pressure to give the title compound as a brown foam (96 mg).

APCl MS m/z 395 [MH]⁺, 417 [MNa]⁺

¹H NMR (400 MHz, CD₃OD): δ 2.00 (m, 2H), 2.27 (m, 1H), 2.58 (m, 1H), 3.11 (s, 3H), 3.36 (m, 1H), 3.62 (m, 2H), 4.21 (m, 4H), 4.40 (m, 1H), 4.55 (m, 1H), 7.00 (t, 1H), 7.44 (d, 1H), 7.88 (m, 2H), 7.92 (m, 2H), 8.06 (t, 1H)

Found; C, 44.30; H, 5.52; N, 14.65; C₂₁H₂₃N₆Cl 0.33CH₂Cl₂. 3HCl. 2.5H₂O requires; C, 44.37; H, 5.53; N, 14.53%.

1. A pharmaceutical composition comprising: (A) a PDE inhibitor, or a pharmaceutically acceptable derivative thereof, and (B) a vasopressin receptor family antagonist, or a pharmaceutically acceptable derivative thereof.

2. The composition according to claim 1, wherein (A) is a PDE5 inhibitor.

3. The composition according to claim 2, wherein the PDE5 inhibitor is sildenafil, tadalafil, vardenafil, DA-8159, or 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one.

4. The composition according to claim 1, wherein (B) is a V₁ₐ receptor antagonist.

5. The composition according to claim 1, wherein (B) is relcovaptan, atosiban, conivaptan, OPC21268, or 8-chloro-5-methyl-1-(3,4,5,6-tetrahydro-2H-[1 ,2′]bipyridinyl-4-yl)-5,6-dihydro-4H-2,3,5,10b-tetraazo-benzo[e]azulene, or a pharmaceutically acceptable salt or solvate thereof.

6. A method of treating dysmenorrhoea comprising administering to a subject in need of such treatment therapeutically effective amounts of: (A) a PDE inhibitor, or a pharmaceutically acceptable derivative thereof, and (B) a vasopressin receptor family antagonist, or a pharmaceutically acceptable derivative thereof, or a pharmaceutical composition comprising such combination.

7. A method according to claim 6, wherein the dysmenorrhea is primary dysmenorrhea.

8. A method according to claim 6, wherein the dysmenorrhea is secondary dysmenorrhea.

9. A method according to claim 8, wherein the secondary dysmenorrhea is a consequence of increased uterine tone.

10. The method according to claim 6, wherein (A) is a PDE5 inhibitor.

11. The method according to claim 10, wherein the PDE5 inhibitor is sildenafil, tadalafil, vardenafil, DA-8159, or 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one.

12. The method according to claim 6, wherein (B) is a V₁a receptor antagonist.

13. The method according to claim 6, wherein (B) is relcovaptan, atosiban, conivaptan, OPC21268, or 8-chloro-5-methyl-1-(3,4,5,6-tetrahydro-2H-[1,2′]bipyridinyl-4-yl)-5,6-dihydro-4H-2,3,5,10b-tetraazo-benzo[e]azulene, or a pharmaceutically acceptable salt or solvate thereof.

14. The method according to claim 6, wherein the administration of (A) and (B) is simultaneous, sequential or separate.
